(19) United States
(12) Patent Application Publication
(10) Pub. No.: US 2008/0138801 A1
(43) Pub. Date: Jun. 12, 2008

(54) Surface Plasmon Resonance Sensor for Detecting Changes in Polynucleotide Mass

(75) Inventor: Liyan He, Melbourne, FL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Arizona Board of Regents, a body corporate acting on behalf of Arizona State University

(21) Appl. No.: 11/578,475

(22) PCT Filed: May 27, 2005
(86) PCT No.: PCT/US05/18958

§ 371 Date: Aug. 14, 2007

Related U.S. Application Data
(60) Provisional application No. 60/575,316, filed on May 28, 2004.

(51) Int. Cl. C12Q 1/68

(52) U.S. Cl. 435/6

(57) ABSTRACT

The present invention provides methods for detecting nucleic acids by generating mass changes in a ligand on an SPR surface through target-dependent enzymatic reaction.

### CROSS REFERENCE

This application claims priority to U.S. Provisional Patent Application Ser. No. 60/575,316 filed May 28, 2004, which is incorporated by reference herein in its entirety.

### FIELD OF INVENTION

This invention relates generally to methods for nucleic acid detection.

### BACKGROUND OF THE INVENTION

Surface plasmon resonance (SPR) reflectivity measurements are surface-sensitive, spectroscopic methods that can be used to characterize the thickness and/or index of refraction of ultrathin organic and biopolymer films at noble metal (Au, Ag, Cu) surfaces. SPR is a physical process that can occur when plane-polarized light hits a metal film under total internal reflection conditions. When a light beam hits a half circular prism, the light is bent towards the plane of interface, when it is passing from a denser medium to a less dense one. Changing the incidence angle changes the out coming light until a critical angle is reached. At this point all the incoming light is reflected within the circular prism. This is called total internal reflection (TIR).

Although no light is coming out of the prism in TIR, the electrical field of the photons extends about a quarter of a wave length beyond the reflecting surface. Now the prism is coated with a thin film of a noble metal on the reflection site. In most cases gold is used because it gives an SPR signal at convenient combinations of reflectance angle and wavelength. In addition, gold is chemically inert to solutions and solutes typically used in biochemical contexts. When the energy of the photon electrical field is just right it can interact with the free electron constellations in the gold surface. The incident light photons are absorbed and converted into surface plasmons. The surface plasmon resonance angle mainly depends on the properties of the metal film, the wavelength of the incident light and the refractive index of the media on either side of the metal film. The binding of biomolecules results in the change of the refractive index on the sensor surface, which is measured as a change in resonance angle or resonance wavelength. The change in refractive index on the surface is correspond to the amount of molecules bound (Quinn, J. G. et al.; Development and application of surface plasmon resonance-based biosensors for the detection of cell-ligand interactions.; Analytical Biochemistry; 281: 135-143; (2000); Akimoto, T. et al. Effect of incident angle of light on sensitivity and detection limit for layers of antibody with surface plasmon resonance spectroscopy; Biosens. Bioelectron.; 15: 355-362; (2000)).

Surface plasmon resonance spectroscopy has become widely used in the fields of chemistry and biochemistry to characterize biological surfaces and to monitor binding events. The success of these SPR measurements is primarily due to three factors: (i) with SPR spectroscopy the kinetics of biomolecular interactions can be measured in real time, (ii) the adsorption of unlabeled analyte molecules to the surface can be monitored, and (iii) SPR has a high degree of surface sensitivity that allows weakly bound interactions to be monitored in the presence of excess solution species. Surface plasmon resonance (SPR) has recently gained attention as a label-free method to monitor such events as antibody-antigen binding, DNA hybridization, and protein-DNA interactions⁵⁻¹⁰.

In a typical SPR biosensing experiment, one interactant in the interactant pair (i.e., a ligand or biomolecule) is immobilized on an SPR-active gold-coated glass slide which forms one wall of a thin flow-cell, and the other interactant in an aqueous buffer solution is induced to flow across this surface, by injecting it through this flow-cell. When light (visible or near infrared) is shined through the glass slide and onto the gold surface at angles and wavelengths near the so-called “surface plasmon resonance” condition, the optical reflectivity of the gold changes very sensitively with the presence of biomolecules on the gold surface or in a thin coating on the gold. The high sensitivity of the optical response is due to the fact that it is a very efficient, collective excitation of conduction electrons near the gold surface. The extent of binding between the solution-phase interactant and the immobilized interactant is easily observed and quantified by monitoring this reflectivity change. An advantage of SPR is its high sensitivity without any fluorescent or other labeling of the interactants.

SPR detects the presence of a biopolymer on a chemically modified gold surface by the change in the local index of refraction that occurs upon adsorption. A relationship is found between resonance energy and mass concentration of biochemically relevant molecules such as proteins, sugars and DNA. The SPR signal is therefore a measure of mass concentration at the sensor chip surface.

Current SPR sensing techniques measure mass change resulting from binding of an analyte to a ligand immobilized on the SPR surface. Since SPR is very sensitive to mass change, high background signals are generated from molecules that bind to the SPR surface non-specifically. This has limited the use of SPR to relatively pure samples. Thus, methods are needed to increase the specificity of SPR measurement, and therefore to increase the sensitivity and application range of SPR sensor-based detection methods.

### SUMMARY OF THE INVENTION

The present invention provides methods for detecting nucleic acids by generating mass changes in a ligand on an SPR surface through target-dependent enzymatic reactions. Nucleic acids that bind to the SPR surface non-specifically are not reactive during the enzymatic reactions, and therefore do not generate changes in the SPR signal.

In one aspect, the present invention provides methods for detecting a target nucleic acid in a test sample comprising (a) contacting a first polynucleotide that binds specifically to a nucleic acid target of interest with a test sample under conditions suitable for binding of the first polynucleotide to the nucleic acid target of interest to form a substrate complex if the nucleic acid target is present in the sample, wherein the first polynucleotide is designed to permit immobilization to a surface plasmon resonance (“SPR”) sensor surface; (b) contacting the substrate complex with reagents under suitable conditions to change the mass of the first polynucleotide via an enzymatic reaction only when the nucleic acid target of interest is present in the substrate complex; and (c) detecting an SPR signal change generated by a mass change of the first polynucleotide immobilized on the SPR sensor surface, wherein a change in mass of the first polynucleotide indicates that the nucleic acid target is present in the test sample.

### BRIEF DESCRIPTION OF THE FIGURES

FIG. 1 is a diagram of the methods of the invention using target dependent ligation reaction to increase the mass of the first polynucleotide.

FIG. 2 is a diagram of the methods of the invention using target dependent polymerase extension reaction to increase the mass of the first polynucleotide.

FIG. 3 is a diagram of the methods of the invention using target circularization and replication to increase the mass of the first polynucleotide.

FIG. 4 is a graph showing real time monitoring of DNA hybridization assays using SPR sensors.

FIG. 5 is a graph showing real time monitoring of DNA polymerization assays using SPR sensors.

### DETAILED DESCRIPTION OF THE INVENTION

All references cited are herein incorporated by reference in their entirety.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

The present invention provides compositions and methods for target detection and quantification by detecting mass changes in polynucleotides using surface plasmon resonance (SPR) sensors.

In one aspect, the present invention provides methods for detecting a target in a test sample comprising

(a) contacting a first polynucleotide that binds specifically to a nucleic acid target of interest with a test sample under conditions suitable for binding of the first polynucleotide to the nucleic acid target of interest to form a substrate complex if the nucleic acid target is present in the sample, wherein the first polynucleotide is designed to permit immobilization to a surface plasmon resonance (“SPR”) sensor surface;

(b) contacting the substrate complex with reagents under suitable conditions to change mass of the first polynucleotide via an enzymatic reaction only when the nucleic acid target of interest is present in the substrate complex; and

(c) detecting an SPR signal change generated by a mass change of the first polynucleotide immobilized on the SPR sensor surface, wherein a change in mass of the first polynucleotide indicates that the nucleic acid target is present in the test sample

The present invention thus provides methods for detecting nucleic acids by generating mass changes in the first polynucleotide through target-dependent enzymatic reactions. The first polynucleotide itself is not a substrate of the enzymatic reaction, but only becomes a substrate of the enzymatic reaction when it is bound to the nucleic acid target of interest in the substrate complex. Nucleic acids that bind to the SPR surface non-specifically are not reactive during the enzymatic reactions since they are not present in the substrate complex, and therefore such non-specific binding events do not generate changes in the SPR signal. As a result, the methods provide increased specificity and sensitivity in nucleic acid detection.

In one embodiment, the detection is made while the nucleic acid target remains bound to the first polynucleotide in the substrate complex. This embodiment permits real-time detection. In another embodiment, the detection is made after removing the nucleic acid target from the substrate complex. This embodiment can be used, for example, when end-point detection is desired.

In a preferred embodiment, the binding comprises hybridization of the first polynucleotide with the nucleic acid target.

The term “polynucleotide” as used herein with respect to each aspect and embodiment of the invention refers to DNA or RNA, preferably DNA, in either single- or double-stranded form. Methods for making such polynucleotides are well known in the art, and numerous commercial suppliers of synthetic polynucleotides are available

The first polynucleotide can be of any length suitable for specifically binding to a nucleic acid target of interest, but is preferably at least 15 nucleotides in length. As will be apparent to those of skill in the art, more than one such polynucleotide can be used in the methods of the invention, as exemplified below. Thus, the methods of the claims require the use of a first polynucleotide but encompass the use of any desired number of polynucleotides to carry out the recited methods. Due to the sensitivity of the SPR sensors, a wide range of concentrations and total amounts of the first polynucleotide (and other polynucleotides) can be used. In a preferred embodiment, the first polynucleotide is added slightly in excess of the expected amount of the nucleic acid target in the test sample, to maximize binding events, but to minimize competition between free and bound first polynucleotide for the active sites on SPR surface during immobilization. It is well within the level of skill in the art to modify the concentration of first polynucleotide used in the methods of the invention as appropriate for a given enzymatic reaction, nucleic acid target, and SPR surface capture efficiency.

As used here “specific” binding means that the first polynucleotide preferentially binds to the nucleic acid target of interest even when the target is present in a complex mixture, such as a test sample as discussed below.

The nucleic acid target can be any DNA or RNA (whether single stranded or double stranded, linear or circular) for which detection is desired. In a preferred embodiment, the nucleic acid target comprises DNA. In another preferred embodiment, the nucleic acid target is circularized. The target nucleic acid can be derived from any type of test sample, including but not limited to tissue samples, body fluids, cell lysates, environmental samples, and nucleic acid samples isolated from any of the above.

As discussed below, “contacting” steps (a) and/or (b) can occur on the SPR sensor surface after immobilization of the first polypeptide onto the SPR surface, or it can occur in solution, followed by immobilization on the SPR sensor surface. For example, in embodiments of the invention involving nucleic acid sequence analysis or other real time detection methods, it is preferred that the contacting in step (b) of the method takes place on the SPR sensor. Those of skill in the art can determine whether the specific assay to be performed would be better served by carrying out the “contacting” steps in (a) and/or (b) in solution or on after SPR surface immobilization of the first polynucleotide.

Determining suitable conditions for binding of the first polynucleotide to the target nucleic acid of interest is well within the level of those of skill in the art. For example, specific conditions used for hybridization depend on the length of the polynucleotide probe employed, their GC content, as well as various other factors as is well known to those of skill in the art. (See, for example, Tijssen (1993) Laboratory Techniques in Biochemistry and Molecular Biology—Hybridization with Nucleic Acid Probes part I, chapter 2, “Overview of principles of hybridization and the strategy of nucleic acid probe assays,” Elsevier, N.Y. (“Tijssen”)).

Reagents that can be used to change the mass of the first polynucleotide via an enzymatic reaction include, but are not limited to nucleotides (including nucleotides, deoxynucleotides, and dideoxynucleotides), other polynucleotide primers, RNA and DNA ligases, preferably, ligase that catalyzes the formation of a phosphodiester bond between juxtaposed 5 phosphate and 3 hydroxyl termini of two adjacent oligonucleotides which are hybridized to a complementary target DNA such as E. coli DNA Ligase, Taq ligase, RNA and DNA polymerases, restriction enzymes, and suitable buffers for carrying out the desired enzymatic reaction. Thus, polymerases and ligases are used together with nucleotides, nucleosides, deoxynucleotides, and/or dideoxynucleotides to increase mass of the first polynucleotide in the substrate complex (ie: when bound to its nucleic acid target) via polymerization or ligation, while restriction enzymes can be used to decrease mass of the first polynucleotide in the substrate complex by a cleavage reaction at a target site formed by the binding of the first polynucleotide to the nucleic acid target. It is well within the level of skill of those in the art to determine appropriate reagent conditions for the desired enzymatic reaction.

Preferably, the first polynucleotide is modified to introduce an affinity moiety for immobilizing the first polynucleotide to a functional group on the SPR surface. The affinity moiety can be any group or molecule that binds specifically to the functional group on the SPR surface. Such affinity moieties include, but are not limited to, biotin, amine, and alkanethiols. The affinity moiety can be linked to the first polynucleotide at the 5′ or 3′ end, or at any internal base as long as the linkage does not interfere with the enzymatic reaction that generates a change in mass of the first polynucleotide. Preferably, the affinity moiety is linked through the 5′ or 3′ end of the first polynucleotide, more preferably, through the 5′ end. Such methods are well known to those of skill in the art (see, for example, Direct immobilization of DNA probes for the development of affinity biosensors, Bioelectrochemistry. Apr. 2005 66(1-2): 129-38 and references cited therein).

Any SPR sensor can be used in the methods of the invention so long as it is capable of generating signals for detecting mass changes in the first polynucleotide according to the methods of the invention. The specific SPR sensor used depends on the proposed use. SPR configurations that can be used with the methods of the invention include, but are not limited to grafting coupled systems, optical waveguide systems, and prisms coupled to attenuated total reflection systems. Various techniques have been developed to activate the SPR surface with various functional groups for immobilization of the first polynucleotide; such techniques are well within the level of skill in the art. See, for example, Uchida K, et al., A reactive poly(ethylene glycol) layer to achieve specific surface plasmon resonance sensing with a high S/N ratio: the substantial role of a short underbrushed PEG layer in minimizing nonspecific adsorption. Anal Chem. 2005 Feb. 15; 77(4): 1075-80; Lu et al., Attachment of functionalized poly (ethylene glycol) films to gold surfaces. Langmuir, 2000. 16(4): p. 1711-1718, Nelson, K. E., et al., Surface characterization of mixed self-assembled monolayers designed for streptavidin immobilization. Langmuir, 2001. 17(9): p. 2807-2816, Lu, H. B., et al. Apatite Coated Surface Plasmon Resonance Biosensors. in Sixth World Biomaterials Congress. 2000. Kamuela, Hawaii, USA: Society for Biomaterials, Minneapolis, Minn., and Lu, H. B., et al. Surface Functionalization for Self-referencing Surface Plasmon Resonance Biosensors by RF-plasma-deposited Thin Films and Self-assembled Monolayers. in Sixth World Biomaterials Congress. 2000. Kamuela, Hawaii, USA: Society for Biomaterials, Minneapolis, Minn. In a further embodiment, the SPR sensor is a fiber optic SPR device, such as those described in U.S. Pat. No. 6,466,323 and U.S. Pat. No. 5,835,645, both incorporated by reference herein in their entirety.

As used herein, the phrase “detecting an SPR signal change” means to make surface-sensitive, SPR reflectivity measurements using spectroscopic methods to characterize the thickness and/or index of refraction of the thin organic and/or biopolymer films at noble metal surfaces. In a preferred embodiment, the detecting comprises detecting a change in the local index of refraction that occurs upon adsorption to the SPR sensor. In the present methods, the SPR signal is a measure of mass concentration at the SPR surface. Detecting a mass change thus involves detecting a difference in SPR signal of the first polynucleotide after conducting the recited steps relative to the SPR signal of the first polynucleotide prior to carrying out the recited steps.

In an embodiment, where the SPR sensor is an SPR-active gold-coated glass slide that forms one wall of a thin flow-cell, the first polynucleotide is immobilized on the SPR sensor surface either before or after hybridization to the nucleic acid target of interest, and the other reagents are added in an aqueous buffer solution that is induced to flow across the SPR sensor by injecting them through the flow-cell. Light (visible or near infrared) is shined through the glass slide and onto the gold surface at angles and wavelengths near the so-called “surface plasmon resonance” condition, the optical reflectivity of the gold changes very sensitively with the change in mass of the first polynucleotide within the substrate complex on the gold surface or in a thin coating on the gold. The extent of mass change is thus observed and quantified by monitoring this reflectivity change.

In a preferred embodiment, the change in mass represents an elongation of the first polynucleotide. The elongation reaction can be carried out on the sensor surface so that the reaction can be monitored in real-time. The elongation reaction can also be performed in solution and the sensor is used to measure the product in an end-point assay. Any reaction that yields polynucleotide elongation can be employed in the disclosed invention, including but not limited to DNA polymerization, RNA polymerization, DNA chain termination reactions, rolling circle replication (RCA), and target mediated ligation. The only prerequisite is that the elongation reaction from the first polynucleotide requires it to be bound to the nucleic acid target in the substrate complex.

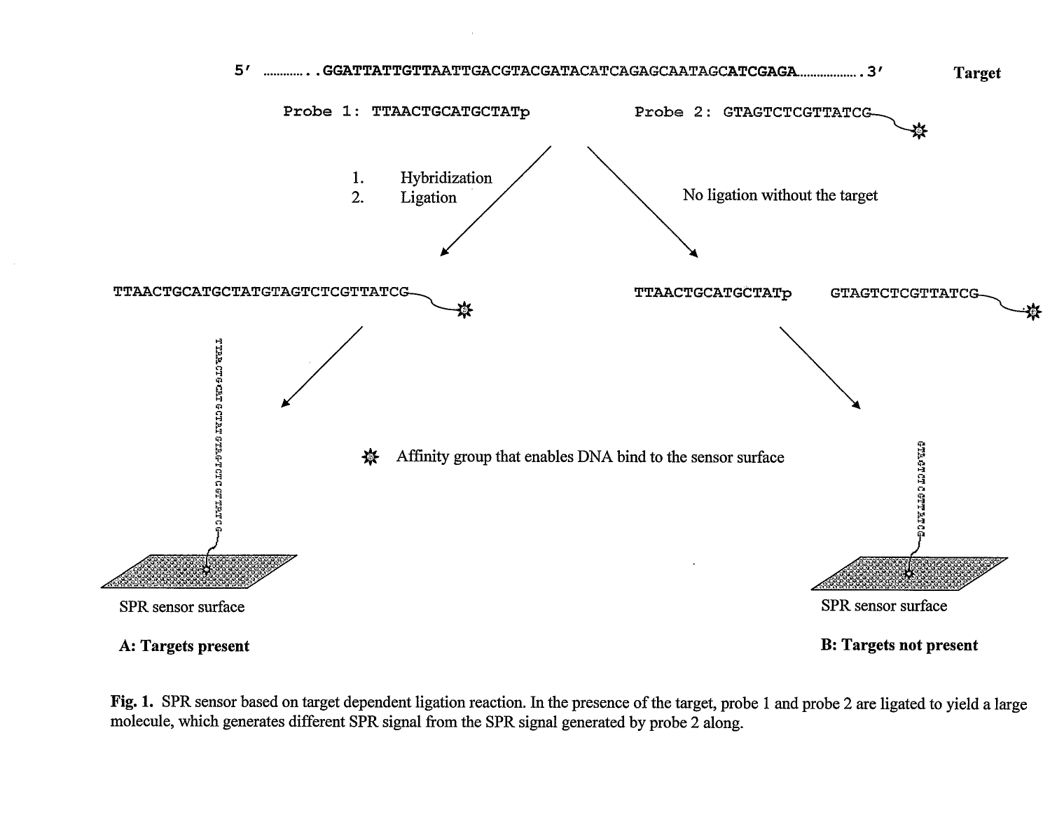

In an exemplary embodiment, elongation is accomplished by a target dependent ligation reaction (FIG. 1). In this embodiment, the method comprises use of target specific DNA probes 1 and 2. Probe 1 is phosphorylated at the 5′ end and it hybridizes to the nucleic acid target. Probe 2, which is the first polynucleotide, is linked to an affinity group at the 5′ end that enables the immobilization of probe 2 on the SPR sensor surface. Probe 2 hybridizes to the nucleic acid target downstream of Probe 1. The method further comprises use of a ligase that catalyzes the formation of a phosphodiester bond between juxtaposed 5′ phosphate and 3′ hydroxyl termini in duplex DNA or RNA, such as Taq DNA ligase. The method further comprises use of an SPR sensor. The detection process includes the steps of (i) Mixing probe 1, probe 2, and the ligase with the sample to be tested; (ii) adding the reaction mixture to the SPR sensor surface under conditions to promote immobilization of Probe 2 to the SPR sensor surface and measuring SPR signal. In the presence of the target, probe 1 and probe 2 are ligated to yield a large molecule, which generates a different SPR signal from the SPR signal generated by probe 2 alone. Alternatively, Probe 2, the first polynucleotide, can be immobilized to the SPR surface first. Probe 1, the sample and ligase are then added and the ligation reaction can be monitored by SPR in real-time. In the presence of the target, Probe 1 is ligated to the first polynucleotide immobilized on the SPR surface, which generates an SPR signal, while ligation will not occur in the absence of the target.

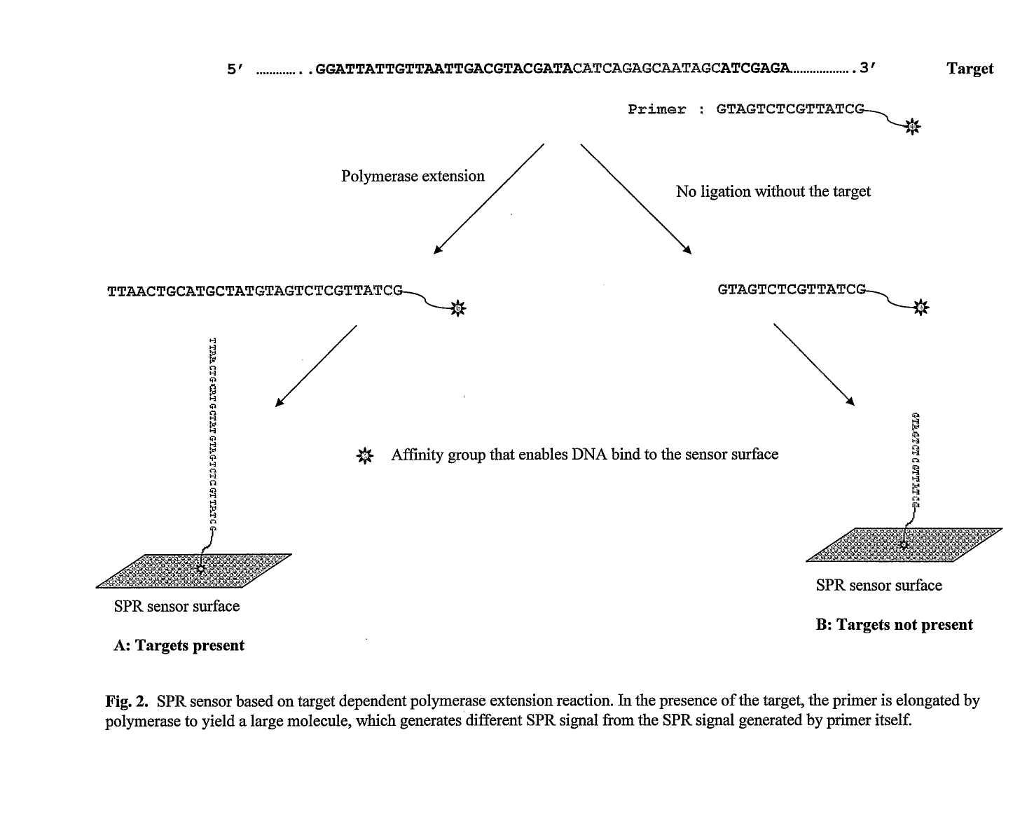

In another example, elongation is accomplished by a target dependent polymerase extension reaction (FIG. 2). The disclosed method comprises use of a target specific DNA polynucleotide (the first polynucleotide), which is linked to an affinity group at the 5′ end that enables the immobilization of polynucleotide on the SPR sensor surface, in combination with a polynucleotide polymerase and an SPR sensor. The detection process includes the steps of (i) mixing the polynucleotide with the sample to be tested; (ii) adding the reaction mixture to the SPR sensor surface under conditions to promote binding of the polynucleotide to the SPR sensor surface, adding polymerase and measuring the SPR signal. In the presence of the target, the target will serve as template so that the first polynucleotide is extended by the polymerase to yield a larger molecule, which generates a detectably different SPR signal from the SPR signal generated by the polynucleotide itself.

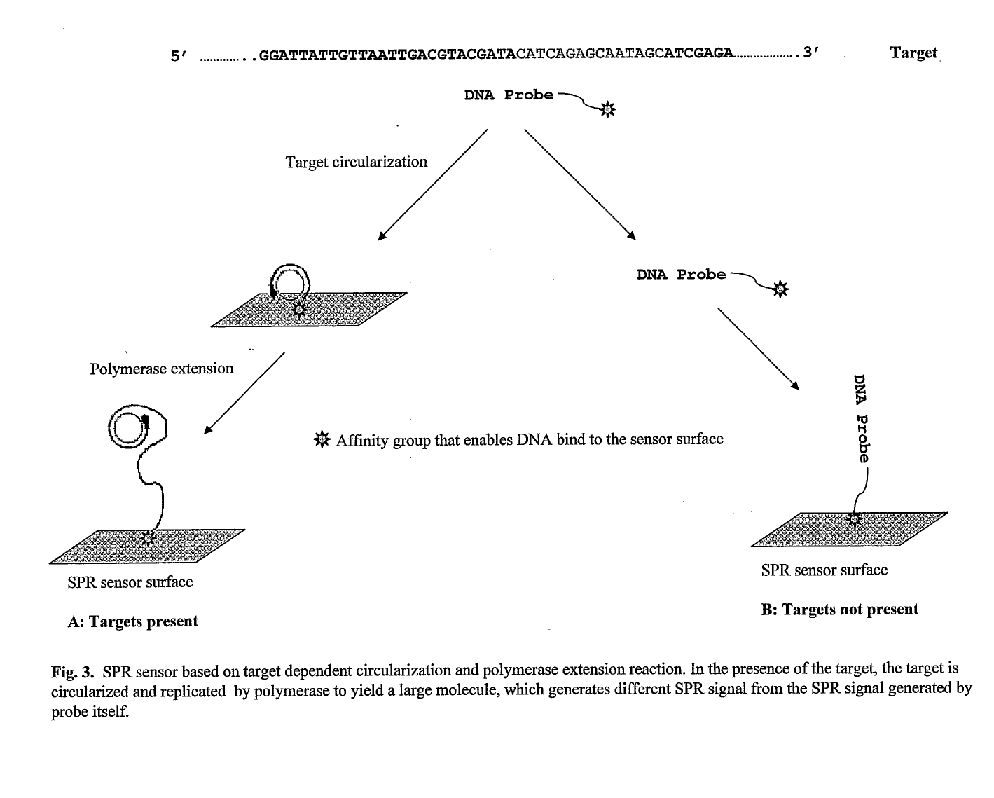

In a further example, the elongation is accomplished by target circularization and replication (FIG. 3). The method includes five components: a double-stranded DNA probe that contains an affinity tag at the 5′ end of the second strand, a polynucleotide ligase, a polymerase, a mixture of nucleotides and an SPR sensor. The detection process comprises the steps of:
1) Circularizing the DNA or RNA target with the double-stranded DNA polynucleotide via any circularization method;
2) Immobilizing the circularized target to the sensor surface through the affinity tag on the double-stranded DNA polynucleotide; and
3) Adding DNA polymerase and a nucleotide mixture to initiate a polymerase reaction using the immobilized DNA strand as primer and the circularized DNA as template. The growth of the immobilized DNA strand is monitored by SPR in real-time.
4) In the absence of the target, no circular DNA will be formed and therefore the immobilized DNA polynucleotide can't be elongated.

In a further embodiment, the methods of the invention can be used for label-free, real-time nucleic acid sequencing, by monitoring changes in mass of the first polynucleotide caused by the template dependent incorporation of nucleotides to nucleic acid target immobilized on the SPR surface via interaction with the first polynucleotide. In a preferred embodiment, these nucleic acid sequencing methods comprise:
(1) Attaching the 5′ end of the first polynucleotide with known sequence to the SPR surface;
(2) Under conditions that are appropriate for nucleic acid hybridization, contacting the immobilized first polynucleotide with the nucleic acid target, in which one part of the nucleic acid target sequence is complementary to the immobilized nucleic acid and the other part of the nucleic acid target sequence is unknown;
(3) Immobilizing the nucleic acid target to the surface via hybridization with the first polynucleotide to form an immobilized nucleic acid partial duplex (the substrate complex) (ie: at the region of complementarity between the first polynucleotide and the nucleic acid target), and wash away the unbound nucleic acid; recording baseline SPR signal;
(4) Contacting the immobilized nucleic acid partial duplex with a nucleotide in the presence of a polymerase under conditions suitable to promote incorporation of the nucleotide into the nucleic acid partial duplex at the 3′ end of the first polynucleotide present in the nucleic acid partial duplex;
(5) Recording sample SPR signal and comparing with the baseline signal. If the polymerase incorporates the added nucleotide to the first nucleic acid, a signal change is observed;
(6) Washing away unincorporated nucleotide;
(7) Repeating steps (4) to (6) a desired number of times with other nucleotides (one nucleotide at a time). The order of nucleotide incorporations reveals the sequence of the unknown sequence in the second nucleic acid.

In a preferred embodiment of the sequencing methods, nucleotide incorporation is initiated by sequentially flowing mixtures of polymerase with different nucleotide through the SPR surface to which the nucleic acid duplex is bound via the first polynucleotide. In a further preferred embodiment, the method is automated.

In another embodiment, the nucleic acid target is a series of fragments, such as restriction enzyme digestion fragments. In this embodiment, it is preferred that different restriction enzyme digest of the nucleic acid target are made to generate a series of overlapping nucleic acid target fragments, to assist in orienting the identified sequence to the nucleic acid target as a whole.

In a further preferred embodiment, nucleotide incorporation is initiated by sequentially merging the SPR surface (preferably in a fiber optic SPR format) containing the immobilized nucleic acid partial duplex into mixtures of polymerase with different nucleotides.

In various other preferred embodiments, the nucleic acid target is a mixture of restriction fragments; the second nucleic acid is circularized; and/or a collection of polynucleotides with different sequences is immobilized at different locations on the SPR surface as a high density array. Such high density arrays can include, but are not limited to, (a) arrays with different polynucleotides, but all complementary to the same target—for example, to facilitate sequence analysis and SNP analysis; (b) arrays with different polynucleotides and with different locations on the array having polynucleotides complementary for different target nucleic acids—for example, to detect different targets nucleic acids; and (c) different sequences immobilized on a sensor array comprising a collection of nanosensors with each nanosensor containing nucleic acids of the same sequence.

Using the methods of the present invention, as little as an atto-mole of DNA target can be detected in a few minutes, as demonstrated in the Example that follows. The methods of the invention have the potential to detect target from crude cell lysate without purification or amplification. Sensor arrays based on the methods of the invention can be used to detect multiple samples simultaneously. Potential applications for the present invention include, but are not limited to, label-free, real time nucleic acid sequencing, high-speed whole genome expression profiling, and ultra fast and sensitive hand-held nucleic acid testing kits.

### EXAMPLES

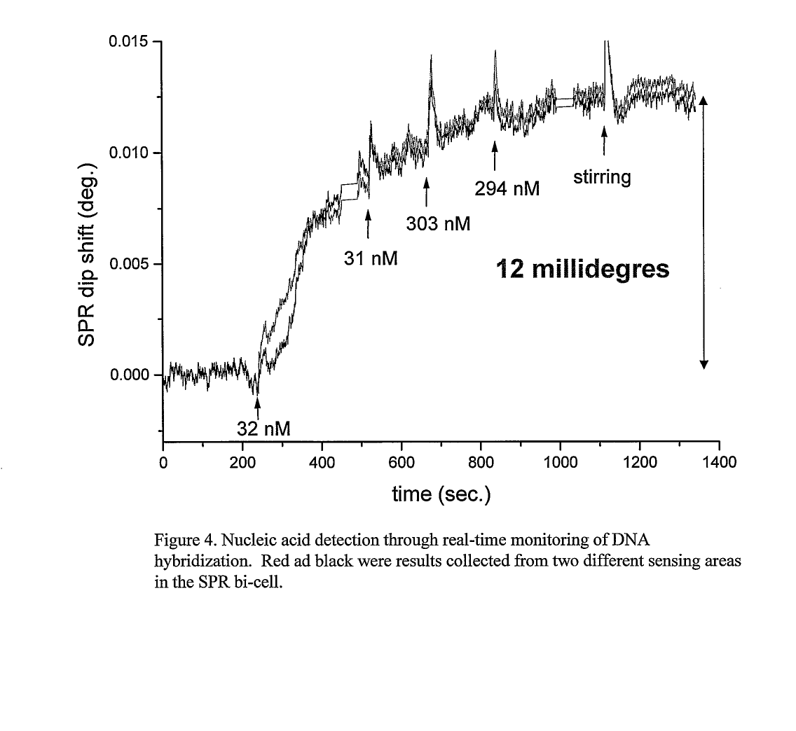

Hybridization assay: 300 ul Phi29 polymerase buffer containing dNTP mixture was added to an SPR bi-cell (Song et al., Nucleic Acids Res. 2002 Jul. 15; 30(14): e72) with an avidin coated gold surface. 28 nmole of 5′ biotinylated DNA probe (the first polynucleotide) was added to the bi-cell. After a 15 min incubation at room temperature to allow DNA probe immobilization to the SPR surface, various amounts of DNA probe complementary sequence (the nucleic acid target) were added while SPR signal was collected continuously using procedures described previously (Song et al., Nucleic Acids Res. 2002 Jul. 15; 30(14): e72). SPR signal increased due to hybridization of the target to the immobilized first polynucleotide. As shown in FIG. 4, addition of 9.6 pmole target generated 7.5 millidegrees SPR dip shift. The signal flatted out after addition of a total of 1.68 nmole target with a total of 12 millidegrees SPR dip shift.

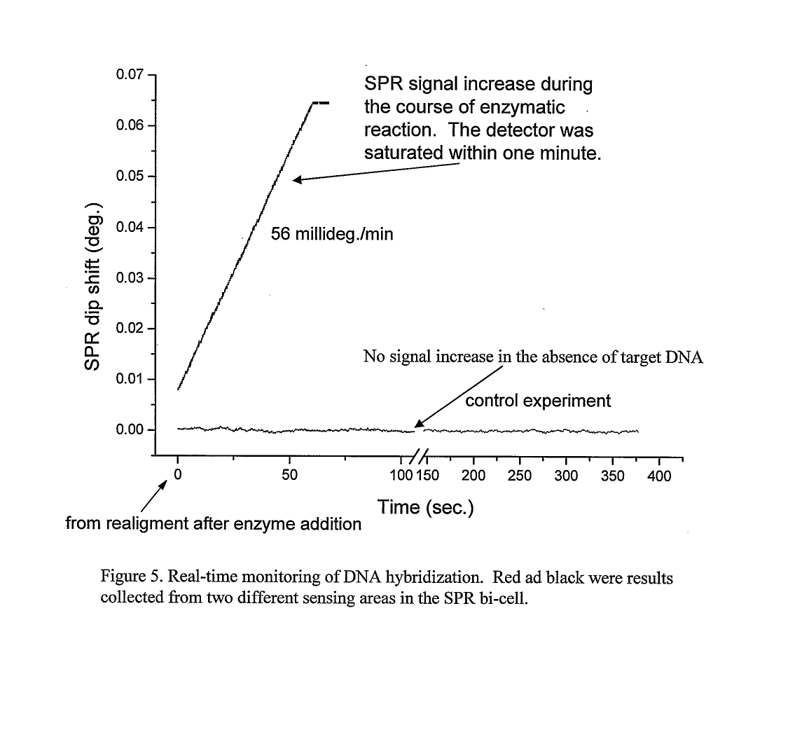

Polymerization assay: 300 ul Phi29 polymerase buffer containing a dNTP mixture was added to an SPR bi-cell (Song et al., Nucleic Acids Res. 2002 Jul. 15; 30(14): e72) with an avidin coated gold surface. Substrate complex, comprising 40 attomole circular DNA target and 160 attomole 5′ biotinylated first polynucleotide hybridized to the target, were added to the bi-cell. After a 15 min incubation at room temperature to allow substrate complex being immobilized to the SPR surface, phi29 polymerase was then added while SPR signal was collected continuously using procedures described before (Song et al., Nucleic Acids Res. 2002 Jul. 15; 30(14): e72). Upon addition of polymerase, SPR signal drafted due to reflective index change caused by the enzyme storage buffer. The detector was realigned and a linear signal increase was observed. The detector was saturated again after 100 second. Signal increase generated by the enzymatic reaction was 56 millidegree/min (black line in FIG. 5). The same experiment was repeated without the DNA target. No signal change was observed in the absence of the DNA target (red line in FIG. 2).

In the hybridization assay (FIG. 4), 9.6 picomole target generated 7.5 millidegrees SPR dip shift. 40 attomole circular DNA target generated more than 50 millidegrees SPR dip shift in one minute through polymerase reaction, while no SPR signal changes was observed in the absence of the target. Therefore, the SPR detection method described in the present invention, which generates SPR signal through enzymatic reaction, is much more sensitive and specific than the current DNA hybridization based SPR method.

### References:

(1) Jordan, C. E.; Frutos, A. G.; Thiel, A. J.; Corn, R. M. Anal. Chem. 1997, 69, 4939-4947.
(2) Peterlinz, K. A.; Georgiadis, R. M. J. Am. Chem. Soc. 1997, 119, 3401-3402.
(3) He, L.; Musick, M. D.; Nicewarner, S. R.; Salinas, F. G.; Benkovic, S. J.; Natan, M. J.; Keating, C. D. J. Am. Chem. Soc. 2000, 122, 9071-9077.
(4) Kai, E.; Sawata, S.; Ikebukuro, K.; Iida, T.; Honda, T.; Karube, I. Anal. Chem. 1999, 71, 796-800.
(5) Brockman, J. M.; Frutos, A. G.; Corn, R. M. J. Am. Chem. Soc. 1999,121, 8044-8051.
(6) Frutos, A. G.; Brockman, J. M.; Corn, R. M. Langmuir 2000, 16, 2192-2197.
(7) Bondeson, K.; Frostellkarlsson, A.; Fagerstam, L.; Magnusson, G. Anal. Biochem. 1993, 214, 245-251.
(8) Babic, I.; Andrew, S. E.; Jirik, F. R. Mutat. Res. 1996, 372, 87-96.
(9) Gotoh, M.; Hasebe, M.; Ohira, T.; Hasegawa, Y.; Shinohara, Y.; Sota, H.; Nakao, J.; Tosu, M. Gen. Anal.: Biomol. Eng. 1997, 14, 47-50.
(10) Lin, S.; Long, S.; Ramirez, S. M.; Cotter, R. J.; Woods, A. S. Anal. Chem. 2000, 72, 2635-2640.

I claim:

1. A method for detecting a target nucleic acid in a test sample, comprising:
(a) contacting a first polynucleotide that binds specifically to a nucleic acid target of interest with a test sample under conditions suitable for binding of the first polynucleotide to the nucleic acid target of interest to form a substrate complex if the nucleic acid target is present in the sample, wherein the first polynucleotide is designed to permit immobilization to a surface plasmon resonance (“SPR”) sensor surface;
(b) contacting the substrate complex with reagents under suitable conditions to change mass of the first polynucleotide via an enzymatic reaction only when the nucleic acid target is present in the substrate complex; and
(c) detecting an SPR signal change generated by a mass change of the first polynucleotide bound to the SPR sensor surface, wherein a change in mass of the first polynucleotide indicates that the nucleic acid target is present in the test sample.

2. The method of claim 1 wherein the enzymatic reaction comprises elongation of the first polynucleotide.

3. The method of claim 1 wherein the enzymatic reaction comprises target dependent ligation of the first polynucleotide to a second polynucleotide that is also complementary to the nucleic acid target.

4. The method of claim 1 wherein the enzymatic reaction comprises nucleic acid polymerization.

5. The method of claim 1 wherein the enzymatic reaction comprises a restriction enzyme cleavage of the first polynucleotide bound to the nucleic acid target.

6. The method of claim 1 wherein the detecting comprises real time detection of a nucleic acid sequence of the nucleic acid target.

7. The method of claim 1 wherein the contacting in step (a) is carried out on the SPR sensor surface.

8. The method of claim 1 wherein the contacting in step (a) is carried out in solution.

9. The method of claim 1 wherein the contacting in step (b) is carried out on the SPR sensor surface.

10. The method of claim 1 wherein the contacting in step (b) is carried out in solution.

11. The method of claim 1 wherein the detecting comprises real time detection of the mass change of the first polynucleotide.

12. The method of claim 1 wherein the detecting is carried out while the first polynucleotide is bound to the nucleic acid target.
